(19) United States
(12) Patent Application Publication
(10) Pub. No.: US 2026/0109937 A1
(43) Pub. Date: Apr. 23, 2026

(54) ENGINEERED MICROORGANISMS WITH ENHANCED PROTEIN EXPRESSION AND SECRETION

(71) Applicant: North Carolina State University, Raleigh, NC (US)

(72) Inventors: Nathan Crook, Raleigh, NC (US); Deniz Durmusoglu, Raleigh, NC (US); Ibrahim Al'Abri, Raleigh, NC (US); Daniel Haller, Raleigh, NC (US)

(21) Appl. No.: 19/114,168

(22) PCT Filed: Sep. 22, 2023
(86) PCT No.: PCT/US2023/074893

§ 371 Date: Mar. 21, 2025

Related U.S. Application Data
(60) Provisional application No. 63/376,912, filed on Sep. 23, 2022.

(51) Int. Cl. C12N 1/18; C12N 9/90; C12R 1/85

(52) U.S. Cl. CPC C12N 1/18; C12N 9/90; C07K 2319/036; C12R 2001/85; C12Y 504/02007

(57) ABSTRACT

The present disclosure provides materials and methods related to the production of peptides and polypeptides from engineered microorganisms. In particular, the present disclosure provides compositions and methods for producing a genetically modified microorganism (e.g., yeast cell) with enhanced protein expression and/or secretion.

### CROSS REFERENCE TO RELATED APPLICATIONS

This application claims priority to and the benefit of U.S. Provisional Patent Application No. 63/376,912 filed Sep. 23, 2022, which is incorporated herein by reference in its entirety and for all purposes.

### GOVERNMENT FUNDING

This invention was made with government support under grant number CBET1934284 awarded by the National Science Foundation. The government has certain rights in the invention.

### SEQUENCE LISTING STATEMENT

The contents of the electronic sequence listing titled (NCSU-41009.601.xml; Size: 3,633 bytes; and Date of Creation: Sep. 22, 2023) is herein incorporated by reference in its entirety.

### FIELD

The present disclosure provides materials and methods related to the production of peptides and polypeptides from engineered microorganisms. In particular, the present disclosure provides compositions and methods for producing a genetically modified microorganism (e.g., yeast cell) with enhanced protein expression and/or secretion.

### BACKGROUND

Engineered probiotics provide unique strategies for disease prevention and treatment. Engineered probiotics are microorganisms that are genetically engineered to stably deliver biotherapeutics, detect disease markers, or perform beneficial functions in the gut environment. Probiotics can be genetically engineered to convert nutrients available in the gut environment to therapeutics or supplements that are not readily available for the host. Additionally, they can be engineered to eliminate metabolic waste or toxins. Recently, probiotics have been engineered to target pathogens such as Clostridioides difficile, treat metabolic diseases such as Phenylketonuria or deliver biotherapeutics such as interleukin-10 to treat inflammatory bowel disease. Furthermore, engineered probiotics can sense and respond to their environment, improving targeting and dosage of therapeutics. Engineered probiotics can potentially be used as a drug delivery platform for many current therapies, target new diseases or treat infections in the gut environment. While all engineered probiotics that are currently in clinical trials are bacterial probiotics, yeast probiotics are currently investigated as a candidate for other applications. However, one challenge in the development of engineered probiotics for use in human patients is control over dosage. If expression of a therapeutic is constitutive (i.e., “always on”) dosage of the therapeutic becomes difficult to control. Additionally, biosynthesis of a therapeutic molecule is often energy- and nutrient-intensive, potentially decreasing the fitness of the engineered probiotic within the gut microbiota, thereby reducing colonization. Therefore, it would be advantageous to control the timing and level of expression of a therapeutic product in S. boulardii in order to control dosage.

### SUMMARY

Embodiments of the present disclosure include an engineered yeast cell comprising (i) at least one genetic modification that produces a functional phosphoglucomutase (PGM2) protein; and (ii) at least one exogenous polynucleotide operably linked to a pGAL1 promoter. In accordance with these embodiments, expression of a target polypeptide or protein encoded by the exogenous polynucleotide is increased compared to a yeast cell lacking the genetic modification.

In some embodiments, the genetic modification comprises a mutation in the gene encoding PGM2. In some embodiments, the at least one genetic modification comprises a point mutation at position 1276, 1277, and/or 1278 in the gene encoding PGM2 (see SEQ ID NO: 1). In some embodiments, the point mutation is: (i) T1276A, T1276C, or T1276G; (ii) G1277C or G1277T; or (iii) A1278T, A1278C, or A1278G.

In some embodiments, the yeast cell is selected from the group consisting of S. boulardii, S. cerevisiae, P. pastoris, Y. lipolytica, C. albicans, K. marxianus, Debaryomyces hansenii, and Kluyveromyces lactis.

In some embodiments, genetic modification enables the yeast cell to utilize galactose as a carbon source. In some embodiments, the genetic modification enhances colonization in a host. In some embodiments, the genetic modification enhances colonization in a host in the presence of galactose.

In some embodiments, the genetic modification activates the pGAL1 promoter. In some embodiments, expression of the target polypeptide or protein encoded by the exogenous polynucleotide is increased compared to a yeast cell lacking the genetic modification.

In some embodiments, the engineered yeast cell further comprises at least one gene knockout. In some embodiments, expression and/or secretion of the target polypeptide or protein is increased at least 1.2-fold compared to a yeast cell lacking the at least one gene knockout. In some embodiments, the genetic knockout comprises a knockout of YPS1 (Accession No. P32329), PRB1 (Accession No. P09232), PEP4 (Accession No. P07267), and/or APE1 (Accession No. P14904). In some embodiments, the genetic knockout comprises a knockout of PRB1 and PEP4. In some embodiments, the genetic knockout comprises a knockout of YPS1, PRB1, PEP4, and APE1.

In some embodiments, the engineered yeast cell comprises a target polypeptide or protein that is at least one of an anti-cancer agent, an immune regulator, an anti-pathogenic agent, and/or a metabolic regulator. In some embodiments, the target polypeptide or protein is at least one of a DARPin, a lectin, a monoclonal antibody, a F(ab′)₂ fragment, a Fab′/Fab fragment, a diabody, a scFv, a nanobody, and/or an affibody.

In some embodiments, the exogenous polynucleotide comprises a secretion signal upstream of the target polypeptide or protein. In some embodiments, the secretion signal comprises an alpha mating factor secretion signal, an invertase secretion signal, a YAP3-TA57 secretion signal, a preOST1-proαMF (I) secretion signal, or a preOST1-proαMF (MUT1) secretion signal, and any combinations thereof.

In some embodiments, the yeast cell is from an S. boulardii strain. In some embodiments, the yeast cell is from an S. boulardiiΔura3 strain (DD277). In some embodiments, the yeast cell is from an S. boulardiiΔura3Δhis3 (DD313) strain. In some embodiments, the yeast cell is from an S. boulardiiΔura3Δhis3PGM2 (DJH077) strain. In some embodiments, the yeast cell is from a strain with an accession number of MYA-796 or a MYA-797.

Embodiments of the present disclosure also include a composition comprising any of the engineered yeast cells described herein. In some embodiments, the composition is lyophilized. In some embodiments, the composition is in wet form. In some embodiments, the composition is in frozen form. In some embodiments, the composition is formulated as a food product.

Embodiments of the present disclosure also include a method of treating and/or preventing a disease or condition in a subject. In accordance with these embodiments, the method includes administering any of the compositions described herein to the subject. In some embodiments, the engineered yeast cells are present in the composition at a dose from about 1×10⁵ cells/kg body weight to about 1×10¹⁵ cells/kg body weight of the subject. In some embodiments, the composition is administered orally or rectally.

Embodiments of the present disclosure also include a method of growing a yeast cell in media comprising galactose. In accordance with these embodiments, the method includes making at least one genetic modification to the yeast cell such that the yeast cell is capable of producing a functional phosphoglucomutase (PGM2) protein. In some embodiments, the yeast cell is a Saccharomyces boulardii cell. In some embodiments, the at least one genetic modification enables the yeast cell to utilize galactose as a carbon source. In some embodiments, the method further comprises inducing expression of an exogenous polypeptide or protein via activation of a pGAL1 promoter. In some embodiments, the exogenous polypeptide or protein is at least one of an anti-cancer agent, an immune regulator, an anti-pathogenic agent, and/or a metabolic regulator.

Embodiments of the present disclosure also include a method of enhancing colonization of a yeast cell in a host. In accordance with these embodiments, the method includes administering a composition comprising an engineered yeast cell that comprises at least one genetic modification that produces a functional phosphoglucomutase (PGM2) protein. In some embodiments, the yeast cell is a Saccharomyces boulardii cell. In some embodiments, the genetic modification enables the yeast cell to utilize galactose as a carbon source. In some embodiments, the engineered yeast cell further comprises at least one exogenous polynucleotide operably linked to a pGAL1 promoter. In some embodiments, the composition comprises galactose. In some embodiments, the engineered Saccharomyces boulardii cell exhibits enhanced colonization in a host as compared to a wild type Saccharomyces boulardii cell. In some embodiments, the composition comprises galactose and wherein the engineered Saccharomyces boulardii cell exhibits enhanced colonization in a host as compared to a wild type Saccharomyces boulardii cell or compared to the engineered Saccharomyces boulardii cell administered without galactose.

### BRIEF DESCRIPTION OF THE DRAWINGS

FIGS. 1A-1L: Growth and fluorescence of edited Sb strains on various carbon sources. WT Sb MYA796 (DD313) and SbGal⁺ (DJH077) were cultured at 37° C. in CSM media supplemented with 2% glucose (a), 2% galactose (b), 2% xylose (c), 2% lactose (d), 2% and raffinose (e), 2% glucose and 2% galactose (f), 1% glucose and 1% galactose (g), 2% glucose and 0.5% galactose (h), 2% raffinose and 2% galactose (i), 1% raffinose and 1% galactose (j), and 2% raffinose and 0.5% raffinose (k) for 36 hours. OD600 measurements were collected every 10 minutes in BioTek Synergy H1 Plate Reader. GFP expression from pGAL1 in media containing various sugars (1). Saturated pre-cultures were subinoculated into 1 ml of media containing various combinations of glucose (4%), raffinose (2%), or galactose (4%) and grown for four hours at 37° C., then incubated at 4° C. for one hour to facilitate GFP maturation. Both strains contain the reporter under the control of pGAL1 on a high-copy (2u) plasmid with a URA3 selective marker. DJH118 includes the repaired PGM2 gene, while DJH107 does not.

FIGS. 2A-2E: Representative graphical data demonstrating Dose-response curves of inducible systems expressing yeGFP. Each reporter construct is on a high-copy (2u) plasmid with a URA3 selective marker. For systems using heterologous repressors, the repressors are expressed from a low-copy (CEN) plasmid with a HIS3 marker. Strains containing the pCUP1 (a), pTET (c), PLAC (d), pXYL (e) constructs were cultured at 37° C. in CSM media lacking either uracil or uracil and histidine supplemented with glucose (2%) with a range of copper, aTc, IPTG, and xylose concentrations, respectively. Strains containing the pGAL1 (b) construct were cultured at 37° C. in CSM media lacking uracil and supplemented with galactose (2%).

FIGS. 3A-3F: Dose-response curves of inducible systems expressing CaFbfp under aerobic and anaerobic conditions. Systems with highest fold induction (pLAC (a), pXYL (b), pGAL1 (c)) were cultured in at 37° C. in CSM media lacking either uracil or uracil and histidine supplemented with glucose (2%) (except for pGAL1) or raffinose (for pGAL1) and range of xylose, IPTG and galactose concentrations, respectively under aerobic or anaerobic (5% H2, 5% CO2, 90% N2) conditions for 24 hours.

FIGS. 4A-4C: Inducible promoter-mediated surface display in probiotic yeast Sb enabling high-throughput screening of protein-protein interactions. (a) Surface display is achieved by the use of alpha-agglutinin system. When pGAL1 promoter is induced, it expresses both AGA1 and AGA2 proteins from the genome and plasmid, respectively. AGA2 is fused with protein tag (V5) and the protein of interest (SA1, anti-toxin peptide). The display is detected via immuno flowcytometry. Sb-SA1 was cultured in CSM-HIS3 media supplemented with glucose (2%) for uninduced and galactose (2%) for induced conditions for 24 hours. Cells were processed for flow cytometry (b) and confocal microscopy (c) upon incubating with anti-V5 Antibody, FITC.

FIGS. 5A-5B: Orthogonality of the inducible promoters in Sb. (a) Evaluating the cross reactivity among the five inducible promoters driving yeGFP expression when exposed to various inducers. Each system was cultivated in CSM media lacking uracil or both uracil and histidine, and supplemented with 2% raffinose, in addition to the highest tested concentration of each inducer (as detailed in FIG. 2). Color intensity is represented as a ratio of induced promoter activity to that of the reference (uninduced state). (b) A dual-fluorescence experiment demonstrating simultaneous expression of yeGFP and mKate, showcasing the potential of employing multiple inducible promoters within the same system.

FIGS. 6A-6C: Modulation Sb colonization profile and residence time in the mouse gut via addition of inducing sugars. Repairing galactose metabolism in Sb results in an enhanced and broader localization within antibiotic-treated mice, demonstrating a clear link between metabolic optimization and colonization profile. (a) Conventional mice were given an antibiotic cocktail (ampicillin (0.5 mg/mL), gentamicin (0.5 mg/mL), metronidazole (0.5 mg/mL), neomycin (0.5 mg/mL), vancomycin (0.25 mg/mL), sucralose (4 mg/mL)) throughout the experiment. Galactose (2 mg/mL) was administered in water starting from Day 0. 10\hat{ }8 CFUs SbGal+ and Sb strains were given to the mice for 4 days. (b) Fecal samples were collected daily and plated on YPD media containing antibiotics. (c) Colonization profiles of Sb and SbGal+ strains in small intestines (SI), cecum and colon in antibiotic treated mice model. Error bars indicate the SD for 3 mice in each experimental arm.

FIGS. 7A-7D: In situ protein expression in the mammalian gut. SbGal+ provides controlled gene expression in the mouse gut. NanoLuciferase expressed via pGAL1 was used to evaluate the functionality and efficiency of SbGal+ in vivo. (a) Experimental outline for in situ protein expression in the mammalian gut. (b) Detection of NanoLuciferase in fecal matter for two days of SbGal+ and galactose treatment. (c) Localization and distribution of NanoLuciferase throughout distinct sections of the lower GI tract, including the small intestine (SI), cecum, and colon. d Luminescent images represent NanoLuciferase activity within GI tract contents, demonstrating variable expression and spatial distribution across different GI tract regions. Each dot represents a mouse in each experimental arm.

FIGS. 8A-8C: Development and validation of genetic logic gates for precision-controlled protein expression in the gut. pTET and pGAL1 were utilized due to their tight regulation between ‘on’ and ‘off’ states, to establish an AND gate for precise protein expression. Conventional mice were given an antibiotic cocktail (ampicillin (0.5 mg/mL), gentamicin (0.5 mg/mL), metronidazole (0.5 mg/mL), neomycin (0.5 mg/mL), vancomycin (0.25 mg/mL), sucralose (4 mg/mL)) throughout the experiment. Galactose (2 mg/mL) was administered in water starting from Day 0. 10\hat{ }8 CFUs SbGal++pGalTet-NLuc strains were given to the mice for 2 days. On Day 0, galactose and aTc administration began ad libitum in water. Fecal samples were collected daily and plated on YPD media containing antibiotics. On Day 2, mice were sacrificed and tissue samples were collected. Daily fecal matter and tissue content were processed with Promega NanoGlo Assay to detect nanoluciferase activity in the samples. Luminescence values were normalized by CFU values. (a) In vitro expression of NanoLuciferase under varied conditions—absence of inducers, presence of individual inducers, or combination of both inducers—provides a demonstration into the effectiveness of the engineered control system. (b) Comparative evaluation of NanoLuciferase activity under these four conditions in the mouse gut, elucidating the functionality of the genetic logic gate in vivo. (c) Quantitative representation of NanoLuciferase expression across distinct sections of the lower GI tract, including the small intestine (SI), cecum, and colon, signifying the versatility and adaptability of the systems of the present disclosure.

FIGS. 9A-9C: In vitro development of reporter inducible strain in yeast synthetic media and chow mice diet. (a) Sb strain expressing yeGFP under the control of galactose inducible promoter (pGal) was tested in yeast complete synthetic media without uracil (CSM-U) and chow mice diet (Chow) with and without galactose. (b) Sb strain expressing NanoLuciferase under the control of pGal in CSM-U in induced and uninduced conditions. (c) Measurement of NanoLuciferase luminescence levels 5, 120 and 180 minutes after Luciferase substrate addition. Each dot represents a biological replicate.

FIGS. 10A-10B: In vitro development of genetic logic gates in Sb. (a) Sb strain expressing yeGFP under the control of galactose-anhydrotetracycline inducible promoter (pGal-Tet) was tested in yeast complete synthetic media without uracil and histidine (CSM-U-H) and chow mice diet (Chow) with and without galactose. (b) Sb strain expressing NanoLuciferase under the control of pGalTet in CSM-U-H in induced and uninduced conditions. Each dot represents a biological replicate.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “oligonucleotide,” refers to a short length of single-stranded polynucleotide chain. Oligonucleotides are typically less than about 300 residues long (e.g., between 15 and 100), however, as used herein, the term is also intended to encompass longer polynucleotide chains. Oligonucleotides are often referred to by their length. For example, a 24-residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

As used herein, “peptide” and “polypeptide,” unless otherwise specified, generally refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, “isolated polynucleotide” generally refers to a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

As used herein, “sequence identity” generally refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein “expression vector” is a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both. Transcriptional and translational control sequences are DNA regulatory sequences, such as promoters, enhancers, terminators, and the like, that provide for the expression of a coding sequence in a host cell. In eukaryotic cells, polyadenylation signals are control sequences.

As used herein, “secretory signal sequence” is a DNA sequence that encodes a polypeptide (a “secretory peptide” that, as a component of a larger polypeptide, directs the larger polypeptide through a secretory pathway of a cell in which it is synthesized. The larger polypeptide is commonly cleaved to remove the secretory peptide during transit through the secretory pathway.

As used here, the term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

As used herein, “operably linked”, when referring to DNA segments, indicates that the segments are arranged so that they function in concert for their intended purposes, e.g., transcription initiates in the promoter and proceeds through the coding segment to the terminator.

As used herein, a coding sequence is “under the control” of transcriptional and translational control sequences in a cell when RNA polymerase transcribes the coding sequence into mRNA, which is then trans-RNA spliced and translated into the protein encoded by the coding sequence.

As used herein “heterologous” DNA refers to DNA not naturally located in the cell, or in a chromosomal site of the cell. Preferably, the heterologous DNA includes a gene foreign to the cell.

### 2. Compositions and Methods

Saccharomyces boulardii is a leading probiotic candidate for delivery of biotherapeutics to the mammalian gut. As the only eukaryotic probiotic chassis, S. boulardii is suitable for applications that are not attainable by probiotic bacteria. Recently, Sb has been established as an easy to engineer probiotic yeast that can be used for production of small molecules and elimination of pathogens. While Sb is a promising probiotic, precise control of gene expression in the species has not been achieved; such control can play a crucial role in maintaining the fitness and survival of the strain during colonization. Developing inducible gene expression systems that can be tuned via the addition of ligands could play an essential role for production and delivery of biotherapeutics. To bridge the gap between controlled gene expression and maintaining fitness, 5 ligand-responsive gene expression systems were developed for S. boulardii. An Sb strain was created that can utilize galactose, allowing tunable gene expression when galactose is the sole carbon source. While the galactose inducible system is the most studied system in yeast, its uses are limited since the presence of glucose blocks the induction of genes controlled by the galactose promoter (pGAL1). Therefore, experiments were conducted to study the induction profiles of four promoters that are activated in the presence of aTc, IPTG, xylose or copper, respectively. Unlike the galactose promoter, these inducible systems can be activated in the presence of glucose. To determine the titration curves of each promoter, aerobic (yeGFP and mKate) and anaerobic (CaFbFP) reporters were used to measure the expression levels under aerobic and anaerobic conditions using different concentrations of the inducers. AND logic gates were then created that enable gene expression in response to multiple ligand inputs. Finally, these systems were used for applications such as surface display, colonization control, and in vivo monitoring of Sb via the expression of nanoluciferase. This work expands the applicability of Sb in the gut microbiome.

In accordance with the above, embodiments of the present disclosure include an engineered yeast cell comprising (i) at least one genetic modification that produces a functional phosphoglucomutase (PGM2) protein; and (ii) at least one exogenous polynucleotide operably linked to a pGAL1 promoter. In some embodiments, expression of a target polypeptide or protein encoded by the exogenous polynucleotide is increased compared to a yeast cell lacking the genetic modification.

In some embodiments, the genetic modification comprises a mutation in the gene encoding PGM2. In some embodiments, the at least one genetic modification comprises a point mutation at position 1276, 1277, and/or 1278 in the gene encoding PGM2. In some embodiments, the point mutation is: (i) T1276A, T1276C, or T1276G; (ii) G1277C or G1277T; or (iii) A1278T, A1278C, or A1278G. In some embodiments, one or more of these point mutations produces a functional phosphoglucomutase (PGM2) protein such that glucose can be effectively utilized by the engineered yeast cell.

In some embodiments, the yeast cell includes, but is not limited to, S. boulardii, S. cerevisiae, P. pastoris, Y. lipolytica, C. albicans, K. marxianus, Debaryomyces hansenii, and Kluyveromyces lactis. In some embodiments, the yeast cell is from an S. boulardiiΔura3 strain (DD277), an S. boulardiiΔura3Δhis3 (DD313) strain, an S. boulardiiΔura3Δhis3PGM2 (DJH077) strain, a MYA-796 strain, or a MYA-797 strain. Other strains of yeast cells can also be used as would be recognized by one of ordinary skill in the art based on the present disclosure.

In some embodiments, genetic modification enables the engineered yeast cell to utilize galactose as a carbon source. In some embodiments, the genetic modification enhances colonization in a host. In some embodiments, the genetic modification enhances colonization in a host in the presence of galactose.

In some embodiments, the genetic modification activates the pGAL1 promoter. In some embodiments, expression of the target polypeptide or protein encoded by the exogenous polynucleotide that is operably linked to the pGAL promoter is increased compared to a yeast cell lacking the genetic modification.

In some embodiments, the engineered yeast cell further comprises at least one gene knockout. In some embodiments, expression and/or secretion of the target polypeptide or protein is increased at least 1.2-fold compared to a yeast cell lacking the at least one gene knockout. In some embodiments, the engineered yeast cells of the present disclosure include at least one genetic modification in a gene involved in the protein secretion pathway. In some embodiments, the genetic medication results in the reduction of the activity and/or expression of the gene(s) involved in the protein secretion pathway. In some embodiments, the genetic modification is a gene knockout or a loss-of-function mutation. The genetic modification can be in any one of the following genes: APE1, YPS1, PRB1, PEP4, PAH1, DER1, HRD1, OCH1, MNN9, VPS5, VPS17, TDA3, GOS1, and ROX1. In accordance with these embodiments, expression of a target polypeptide or protein is increased compared to a yeast cell lacking at least one genetic modification in one or more of these genes. In some embodiments, the genetic knockout comprises a knockout of YPS1 (Accession No. P32329), PRB1 (Accession No. P09232), PEP4 (Accession No. P07267), and/or APE1 (Accession No. P14904). In some embodiments, the genetic knockout comprises a knockout of PRB1 and PEP4. In some embodiments, the genetic knockout comprises a knockout of YPS1, PRB1, PEP4, and APE1.

In some embodiments, the engineered yeast cell comprises a target polypeptide or protein that is at least one of an anti-cancer agent, an immune regulator, an anti-pathogenic agent, and/or a metabolic regulator. In some embodiments, the target polypeptide or protein is at least one of a DARPin, a lectin, a monoclonal antibody, a F(ab′)₂ fragment, a Fab′/Fab fragment, a diabody, a scFv, a nanobody, and/or an affibody.

In some embodiments, the exogenous polynucleotide comprises other features that facilitate protein expression and/or secretion in the engineered yeast cell. For example, the exogenous polypeptide can include a promoter upstream of the target polypeptide or protein in order to facilitate its expression. In some embodiments, the exogenous polynucleotide can include a secretion signal upstream of the target polypeptide or protein in order to facilitate its secretion from the engineered yeast cell. Although any suitable promoter or secretion signal can be used, as would be recognized by one of ordinary skill in the art, in some embodiments, the secretion signal comprises an alpha mating factor secretion signal. In some embodiments, the target polypeptide or protein is expressed and secreted from the engineered yeast cell. In some embodiments, the target polypeptide or protein is expressed on the surface of the engineered yeast cell. In some embodiments, the exogenous polynucleotide comprises a secretion signal upstream of the target polypeptide or protein. In some embodiments, the secretion signal comprises an alpha mating factor secretion signal, an invertase secretion signal, a YAP3-TA57 secretion signal, a preOST1-proαMF (I) secretion signal, or a preOST1-pro α MF (MUT1) secretion signal, and any combinations thereof.

In some embodiments, the engineered yeast cells of the present disclosure comprising at least one of the genetic modifications described herein exhibit increased expression, secretion, and/or cell surface display of the target polypeptide or protein. In some embodiments, expression, secretion, and/or cell surface display of the target polypeptide or protein is increased at least 1.1-fold compared to a yeast cell lacking the genetic modification. In some embodiments, expression, secretion, and/or cell surface display of the target polypeptide or protein is increased at least 1.2-fold compared to a yeast cell lacking the genetic modification. In some embodiments, expression, secretion, and/or cell surface display of the target polypeptide or protein is increased at least 1.3-fold compared to a yeast cell lacking the genetic modification. In some embodiments, expression, secretion, and/or cell surface display of the target polypeptide or protein is increased at least 1.4-fold compared to a yeast cell lacking the genetic modification. In some embodiments, expression, secretion, and/or cell surface display of the target polypeptide or protein is increased at least 1.5-fold compared to a yeast cell lacking the genetic modification. In some embodiments, expression, secretion, and/or cell surface display of the target polypeptide or protein is increased at least 1.6-fold compared to a yeast cell lacking the genetic modification. In some embodiments, expression, secretion, and/or cell surface display of the target polypeptide or protein is increased at least 1.7-fold compared to a yeast cell lacking the genetic modification. In some embodiments, expression, secretion, and/or cell surface display of the target polypeptide or protein is increased at least 1.8-fold compared to a yeast cell lacking the genetic modification. In some embodiments, expression, secretion, and/or cell surface display of the target polypeptide or protein is increased at least 1.9-fold compared to a yeast cell lacking the genetic modification. In some embodiments, expression, secretion, and/or cell surface display of the target polypeptide or protein is increased at least 2.0-fold compared to a yeast cell lacking the genetic modification. In some embodiments, expression, secretion, and/or cell surface display of the target polypeptide or protein is increased at least 3.0-fold compared to a yeast cell lacking the genetic modification. In some embodiments, expression, secretion, and/or cell surface display of the target polypeptide or protein is increased at least 4.0-fold compared to a yeast cell lacking the genetic modification. In some embodiments, expression, secretion, and/or cell surface display of the target polypeptide or protein is increased at least 5.0-fold compared to a yeast cell lacking the genetic modification. In some embodiments, expression, secretion, and/or cell surface display of the target polypeptide or protein is increased at least 6.0-fold compared to a yeast cell lacking the genetic modification. In some embodiments, expression, secretion, and/or cell surface display of the target polypeptide or protein is increased at least 7.0-fold compared to a yeast cell lacking the genetic modification. In some embodiments, expression, secretion, and/or cell surface display of the target polypeptide or protein is increased at least 8.0-fold compared to a yeast cell lacking the genetic modification. In some embodiments, expression, secretion, and/or cell surface display of the target polypeptide or protein is increased at least 9.0-fold compared to a yeast cell lacking the genetic modification. In some embodiments, expression, secretion, and/or cell surface display of the target polypeptide or protein is increased at least 10.0-fold compared to a yeast cell lacking the genetic modification. In some embodiments, expression, secretion, and/or cell surface display of the target polypeptide or protein is increased at least 15.0-fold compared to a yeast cell lacking the genetic modification. In some embodiments, expression, secretion, and/or cell surface display of the target polypeptide or protein is increased at least 20.0-fold compared to a yeast cell lacking the genetic modification.

In accordance with the above embodiments, the present disclosure also includes a composition comprising any of the engineered yeast cells described herein. In some embodiments, the composition is formulated as a food product or a medicament. In some embodiments, the composition is lyophilized. In some embodiments, the composition is in wet form. In some embodiments, the composition is in frozen form. As would be recognized by one of ordinary skill in the art based on the present disclosure, yeast cells can be formulated as a lyophilized composition (e.g., including a cryoprotectant) and can be readily reconstituted, which is an advantage over many other microorganisms (e.g., bacteria and fungi). In this aspect, the compositions of the present disclosure are particularly suited for lyophilization and formulation as an LBP and/or therapeutic composition for administration to a subject.

In some embodiments, the engineered yeast cells are present in the composition at a dose from about 1×10⁵ cells/kg body weight to about 1×10¹⁵ cells/kg body weight of the subject. In some embodiments, the engineered yeast cells are present in the composition at a dose from about 1×10⁵ cells/kg body weight to about 1×10¹⁴ cells/kg body weight of the subject. In some embodiments, the engineered yeast cells are present in the composition at a dose from about 1×10⁵ cells/kg body weight to about 1×10¹³ cells/kg body weight of the subject. In some embodiments, the engineered yeast cells are present in the composition at a dose from about 1×10⁵ cells/kg body weight to about 1×10¹² cells/kg body weight of the subject. In some embodiments, the engineered yeast cells are present in the composition at a dose from about 1×10⁵ cells/kg body weight to about 1×10¹¹ cells/kg body weight of the subject. In some embodiments, the engineered yeast cells are present in the composition at a dose from about 1×10⁵ cells/kg body weight to about 1×10¹⁰ cells/kg body weight of the subject. In some embodiments, the engineered yeast cells are present in the composition at a dose from about 1×10⁵ cells/kg body weight to about 1×10⁶ cells/kg body weight of the subject. In some embodiments, the engineered yeast cells are present in the composition at a dose from about 1×10⁵ cells/kg body weight to about 1×10⁷ cells/kg body weight of the subject. In some embodiments, the engineered yeast cells are present in the composition at a dose from about 1×10⁵ cells/kg body weight to about 1×10⁸ cells/kg body weight of the subject. In some embodiments, the engineered yeast cells are present in the composition at a dose from about 1×10⁵ cells/kg body weight to about 1×10⁹ cells/kg body weight of the subject. In some embodiments, the engineered yeast cells are present in the composition at a dose from about 1×10⁵ cells/kg body weight to about 1×10¹⁰ cells/kg body weight of the subject. In some embodiments, the engineered yeast cells are present in the composition at a dose from about 1×10¹⁰ cells/kg body weight to about 1×10¹⁵ cells/kg body weight of the subject. In some embodiments, the engineered yeast cells are present in the composition at a dose from about 1×10⁸ cells/kg body weight to about 1×10¹² cells/kg body weight of the subject.

In some embodiments, the composition comprising the engineered yeast cells of the present disclosure further comprises at least one pharmaceutically acceptable excipient or carrier. A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising the engineered yeast cells, a therapeutically effective amount can be initially determined from animal models. A therapeutically effective dose can also be determined from human data which are known to exhibit similar pharmacological activities, such as other adjuvants. The applied dose can be adjusted based on the relative bioavailability and potency of the administered engineered yeast cells and the corresponding proteins or peptides expressed by the engineered yeast cells. Adjusting the dose to achieve maximal efficacy based on the methods described above and other methods as are well-known in the art is well within the capabilities of the ordinarily skilled person in the art.

The compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration. The compositions described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, inhalable, topical, injectable, immediate-release, pulsatile-release, delayed-release, or sustained release). The pharmaceutically acceptable compositions may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20.

Embodiments of the present disclosure also include a method of treating and/or preventing a disease or condition in a subject by administering any of the engineered yeast cells or compositions comprising the engineered yeast cells described herein. In accordance with these embodiments, the methods include administering any of the compositions described herein to the subject. In some embodiments, the composition is administered orally, rectally, parenterally, intramuscularly, intraperitoneally, intravenously, intracerebroventricularly, intracisternally, subcutaneously, via injection or infusion, via inhalation, spray, nasal, vaginal, rectal, sublingual, or topical administration. In some embodiments, the composition is administered orally or rectally. In some embodiments, the engineered yeast cells are present in the composition at a dose from about 1×10⁵ cells/kg body weight to about 1×10¹⁵ cells/kg body weight of the subject. In some embodiments, the composition is administered orally or rectally.

Embodiments of the present disclosure also include a method of growing a yeast cell in media comprising galactose. In accordance with these embodiments, the method includes making at least one genetic modification to the yeast cell such that the yeast cell is capable of producing a functional phosphoglucomutase (PGM2) protein. In some embodiments, the yeast cell is a Saccharomyces boulardii cell. In some embodiments, the at least one genetic modification enables the yeast cell to utilize galactose as a carbon source. In some embodiments, the method further comprises inducing expression of an exogenous polypeptide or protein via activation of a pGAL1 promoter. In some embodiments, the exogenous polypeptide or protein is at least one of an anti-cancer agent, an immune regulator, an anti-pathogenic agent, and/or a metabolic regulator.

Embodiments of the present disclosure also include a method of enhancing colonization of a yeast cell in a host. In accordance with these embodiments, the method includes administering a composition comprising an engineered yeast cell that comprises at least one genetic modification that produces a functional phosphoglucomutase (PGM2) protein. In some embodiments, the yeast cell is a Saccharomyces boulardii cell. In some embodiments, the genetic modification enables the yeast cell to utilize galactose as a carbon source. In some embodiments, the engineered yeast cell further comprises at least one exogenous polynucleotide operably linked to a pGAL1 promoter. In some embodiments, the composition comprises galactose. In some embodiments, the engineered Saccharomyces boulardii cell exhibits enhanced colonization in a host as compared to a wild type Saccharomyces boulardii cell. In some embodiments, the composition comprises galactose and wherein the engineered Saccharomyces boulardii cell exhibits enhanced colonization in a host as compared to a wild type Saccharomyces boulardii cell or compared to the engineered Saccharomyces boulardii cell administered without galactose.

### Materials and Methods

Strains and Culture Media. Escherichia coli NEB Stable, NEB 5α, and NEB 10ß were used for plasmid construction and maintenance. E. coli cells were grown in lysogeny broth (LB) (5 g/L yeast extract, 10 g/L tryptone, 10 g/L NaCl) at 37° C. supplemented with ampicillin (100 μg/mL), kanamycin (50 μg/mL) or chloramphenicol (34 μg/mL). Saccharomyces boulardii ATCC-MYA796ΔURA3ΔHIS3 was used to construct SbGal⁺, and SbGal⁺ was used for subsequent inducible promoter characterization, yeast surface display, and logic gate experiments. Sc BY4741 was used as a control for certain experiments. Yeast cultures for genome editing were grown in yeast extract-peptone-dextrose (YPD) medium (50 g/L YPD Broth (Sigma-Aldrich)). For all other experiments, yeast cultures were grown in synthetic complete media containing 0.67% (w/v) Yeast Nitrogen Base Without Amino Acids (Sigma-Aldrich), 1.92 g/L Yeast Synthetic Media Dropout Mix (uracil, histidine, or both), and glucose (2% (w/v) as a carbon source unless otherwise indicated. All S. boulardii strains were grown at 37° C. and all S. cerevisiae strains were grown at 30° C. Anaerobic cultivations were carried out in an anaerobic chamber (Coy lab) at 37° C. with agitation provided by gentle rocking.

Plasmid and Strain Construction. SbGal⁺ was constructed by introducing a point mutation into the S. boulardii PGM2 gene via CRISPR-Cas9. Primers DJHpr088 and DJHpr089 were used to amplify 302 bp of the S. cerevisiae PGM2 gene for use as a repair template. Plasmid ISA1041 provided guide RNA and Cas9 nuclease to carry the edit.

A synthetic toolkit (MoClo-YTK) containing yeast parts were gifts from the Dueber Lab (Addgene #1000000061). Expression vectors for yeGFP, mKate, and CaFBFP consisted of two connectors, an inducible or constitutive promoter, the fluorescent protein coding sequence, the tENO1 terminator, the URA3 yeast marker, the 2 micron yeast origin, and Amp/ColE1 as an E. coli marker and origin. Similarly, expression vectors for cognate repressor proteins included two connectors, the constitutive promoter pFBA1, the repressor protein coding sequence, the tENO2 terminator, the HIS3 yeast marker, the CEN yeast origin, and Amp/ColE1 as an E. coli marker and origin. All yeast parts were included in the MoClo-YKT kit except for the following parts, which were ordered as gBlocks with appropriate restriction sites and overhangs: pTET, PLAC, pXYL, pFBA1, tetR, lacI, xylR, CaFBFP, and mKate. Expression vectors were assembled according to Deuber lab YTK protocols via Golden Gate cloning, with the Golden Gate reaction mixture containing 0.5 μL of 40 nM of each DNA part (20 fmol), 0.5 μL T7 ligase (EB), 1.0 μL T4 Ligase Buffer (NEB), and 0.5 μL BsaI (10,000 U/mL, NEB), with water to bring the final volume to 10 μL. Assembly was performed on a thermocycler using the following program: 30 cycles of digestion (37° C. for 2 min) and ligation (16° C. for 5 min), followed by a final digestion (60° C. for 10 min) and heat inactivation (80° C. for 10 min).

To construct condensed plasmids for orthogonality experiments, Gibson Assembly was used to assemble both the inducible promoter-fluorescent protein transcriptional unit and constitutive promoter-cognate repressor transcriptional unit into the same backbone, separated by a connector. For each condensed plasmid, three fragments were amplified, consisting of the two transcriptional units and yeast backbone with E. coli marker and origin, with ˜20 bp homology between fragments. The Gibson Assembly mixture consisted of the following: 100 ng backbone fragment, additional insert fragments in 2:1 molar ratio to backbone fragment, 10 μL HiFi 2× Master Mix (NEB), and water up to 20 μL. The reactions were incubated in a thermocycler at 50° C. for 30 min prior to transformation to E. coli.

AGA2-sfGFP plasmid was constructed via Gibson assembly. pYD1 plasmid was gift from the Wittrup Lab (Addgene #xxxxx) and TRP1 marker on pYD1 was swapped with HIS3 marker from MoClo-YTK via 2-part Gibson cloning, resulting in pYD1-HIS3. Then, sfGFP was ordered as gene fragment and was inserted into MCS on pYD1-HIS3 via 2-part Gibson cloning.

pGAL1-AGA1-URA3 integration cassette was constructed via Golden Gate assembly. AGA1 was amplified from Sb genome. pGAL1 (YTK030), amplified AGA1 and tENO1 (YTK051) were inserted in ISA086 via Golden gate cloning.

The nourseothricin resistance gene natR was amplified and assembled into an integration cassette to form ISA186 by Golden Gate assembly as described above. The resulting integration cassette provided the DNA repair template for a CRISPR-Cas12a-based insertion of natR into the SbGal⁺ genome, producing SbGal⁺/NatR.

Integration cassettes for logic gate constructs were constructed via Golden Gate assembly of the following parts: The integration cassette backbone, the logic gate, and any necessary transcriptional units for repressor proteins. All parts were ordered as gBlocks with appropriate restriction sites and overhangs for Golden Gate assembly except for the backbone and the pFBA1-tetR-tENO2 transcriptional unit, which was amplified from an expression vector. For the logic gate experiments, constitutive promoters pPXR1 and pVM46 were used for repressor proteins Lacl and XyIR, respectively.

Yeast Competent Cells and Transformations. A yeast competent cell preparation and transformation protocol from Gietz et. Al was used. To prepare competent cells, yeast colonies were inoculated into 1 mL YPD and incubated in a shaking incubator overnight at 37° C., 250 rpm. This culture was diluted into fresh 25 ml YPD (with OD600=0.25) and grown to OD 0.8-1.0. Cells were pelleted by centrifugation for 5 minutes at 3,000×g and resuspended in 25 ml autoclaved water before being centrifuged again under the same conditions. The cells were then resuspended in 1 mL lithium acetate (100 mM, Sigma-Aldrich) and centrifuged again under the same conditions. The cells were resuspended in 250 μL lithium acetate (100 mM) and divided into transformation tubes, with 50 μL/tube. Cells were washed again in 1 mL lithium acetate (100 mM) before being spun down and the supernatant removed. The cell pellet was then gently resuspended in 50 μL boiled salmon sperm DNA (2 mg/ml), and transformation reagents were added in the following order: 2 μg DNA repair template if applicable, 1 μg of any yeast plasmids (either for expression or for gRNA and Cas12a expression), 36 μL lithium acetate (1.0 M), and 260 μL PEG3350 (50%, Fisher Scientific). To produce the salmon sperm DNA, double-stranded salmon sperm DNA (Invitrogen, 15632011) at 10 mg/ml was diluted to 2 mg/mL and incubated at 95° C. for 5 min to denature the DNA. The transformation mix was gently vortexed for less than 5 seconds at low speed before being heat shocked at 42° C. for one hour. The transformation reactions were then centrifuged for 3 minutes at 3,000×g, and the supernatant was removed and discarded. The cells were resuspended in 1 mL YPD by gentle pipetting, and recovered for 1 hour at 37° C. (or 30° C. for S. cerevisiae). In the case of genome editing reactions, this recovery period was extended to a total of 3 hours. Finally, the cell suspension was centrifuged for 1 minute at 3,000×g and the pellet was resuspended in 100 μL. 50 μL of the suspension was plated on appropriate growth media.

Yeast Genome Editing. For yeast genome editing, the protocol above was followed, with the DNA transformed including 1 μg of a Cas12a-encoding plasmid ISA166, 1 μg of a guide RNA plasmid, and 2 μg of a linear DNA repair template with 350-900 bp of homology to the target site in the genome on each side. Repair templates included the URA3 gene to aid in selection of positive transformants through plating on growth media lacking uracil.

S. boulardii Colony PCR. Yeast genome edits were confirmed using Phire Plant Direct PCR Master Mix from Thermo Fisher. The protocol for performing PCR amplification directly from yeast colonies is described by the manufacturer. Briefly, 10 μL of master mix was combined with 1 μL of each primer and water up to 20 μL. Using a pipette tip, a small part of a yeast colony was picked and resuspended in the PCR reaction. If PCR fails, then use modified protocol. Resuspend small colony in 8 μL of 20 mM NaOH and incubate reaction at 98° C. for 10 minutes then add 10 μL of master mix was combined with 1 μL of each primer to the lysed cells. The PCR reaction then proceeded according to the supplier's specifications. Primers were designed to bind outside the linear repair template's homology arms.

Construction of Growth Curves. Three biological replicates of each strain were grown overnight at 37° C., 250 rpm in their corresponding media. The cultures were then subinoculated to OD 0.1 in 96-well-plates (Costar, Corning™ 3788) in appropriate media and grown for 36 hours in a plate reader (BioTek Synergy™ H1, Shake Mode: Double Orbital, Orbital Frequency: continuous shake 365 rpm, Interval: 10 min).

Flow Cytometry and Dose-Response Curve Construction. Yeast strains were inoculated from single colonies on plates into 1 mL of appropriate media and grown overnight at 37° C. (for S. boulardii) or 30° C. (for S. cerevisiae). Cultures were then subinoculated to OD 0.1 in media containing any appropriate inducer molecules. Cultures were induced for 8 hours unless otherwise specified and incubated at 4° C. for 1-2 hours to facilitate protein folding (anaerobic cultures were not incubated at 4° C.). Cultures were then diluted to OD 0.1-0.5 in flat-bottom 96-well plates and run on a BD Accuri™ C6 Plus Flow Cytometer. For each replicate, 10,000 events were collected under settings of FSCH-H<20,000 and SSC-H<600 and medium to low flow. Fluorescence was detected on the FITC channel for the yeGFP and PerCP channel for the mKate2. No gating was performed.

Dose response curves and orthogonality: Yeast strains were inoculated from single colonies on plates into 1 mL of appropriate media and grown overnight at 37° C. (for S. boulardii). Cultures were then subinoculated to OD 0.1 in media containing any appropriate inducer molecules. Cultures were induced for 24 hours and incubated at 4° C. for 1-2 hours to facilitate protein folding (anaerobic cultures were not incubated at 4° C.). Cultures were then diluted to OD 0.1-0.5 in flat-bottom 96-well plates and run on a BD Accuri™ C6 Plus Flow Cytometer. For each replicate, 10,000 events were collected under settings of FSCH-H<20,000 and SSC-H<600 and medium to low flow. Fluorescence was detected on the FITC channel for the yeGFP, CAFBFP and PerCP channel for the mKate2. No gating was performed. For induced display detection, 10\hat{ }7 cells were processed for flow cytometry analysis. After media removal, cells were incubated either with 50 μL anti-V5 Antibody, FITC or mouse anti-V5 antibody. Cells were collected and washed in 0.1% BSA 1×PBS. Cells incubated with mouse anti-V5 antibody were then labeled with 50 μL secondary antibody goat anti-mouse antibody, AlexaFluor647. Cells were collected and washed in 0.1% BSA 1×PBS. Then, cells with both antibody combinations were resuspended in 200 μL PBS and run on a BD Accuri™ C6 Plus Flow Cytometer in 96-well plate format. For each replicate, 10,000 events were collected under settings of FSCH-H<20,000 and SSC-H<600 and medium flow. Fluorescence was detected on the FITC channel for the FITC and APC channel for the AlexaFluor647. No gating was performed.

Mouse Experiments. All mouse experiments were approved by the NC State University Institutional Animal Care and Use Committee (IACUC). Six-week old female C57BL/6J mice were obtained from Jackson Laboratories and hosted at the NCSU Biological Resources Facility (BRF) for 3-4 days before experiments. Mice were housed in groups of three and their cages were changed before treatment with antibiotic cocktail, sugar administration and before treatment with SbPGM2reversion::NatR or Sb::NatR. Antibiotic cocktail (ampicillin (0.5 mg/mL), gentamicin (0.5 mg/mL), metronidazole (0.5 mg/mL), neomycin (0.5 mg/mL), vancomycin (0.25 mg/mL)), sucralose (4 mg/mL) and galactose (20 mg/mL) were administered ad libitum in filter sterilized drinking water during the experiment and refreshed daily. Antibiotic administration was started 3 days prior to Sb gavage. Mice were gavaged with 10\hat{ }8 CFU Sb (SbPGM2::NatR or Sb:NatR) every day for 3 days. Fecal samples were collected every 24 h from day 1 to day 9. On Day 9, the mice were sacrificed and intestinal contents (small intestine, cecum, colon) were collected.

About 1-2 pieces of stool or intestinal matter were collected in preweighed 1.5 mL centrifuge tubes and then weighed again to determine fecal mass. Fecal matter was then resuspended in 1 mL PBS per 10 mg feces. Fecal suspensions were plated on YPD media containing 50 μg/mL nourseothricin and 0.25 mg/mL streptomycin. Plates were sealed with parafilm and incubated at 37° C. for 2-3 days.

As referenced further herein, the nucleotide sequence of phosphoglucomutase (PGM2) from Saccharomyces boulardii is provided below:

(SEQ ID NO: 1)
ATGTCATTTCAAATTGAAACGGTTCCCACCAAACCATATGAAGACCAAAA
GCCTGGTACCTCTGGTTTGCGTAAGAAGACAAAGGTGTTTAAAGACGAAC
CTAACTACACAGAAAATTTCATTCAATCGATCATGGAAGCTATTCCAGAG
GGTTCTAAAGGTGCCACTCTTGTTGTCGGTGGTGATGGGCGTTACTACAA
TGATGTCATTCTTCATAAGATTGCCGCTATCGGTGCTGCCAACGGTATTA
AAAAGTTAGTTATTGGTCAGCATGGTCTTCTGTCTACGCCAGCCGCTTCT
CACATCATGAGAACCTACGAGGAAAAATGTACTGGTGGTATTATCTTAAC
CGCCTCACATAATCCAGGTGGTCCAGAAAATGACATGGGTATTAAGTATA
ACTTATCCAATGGGGGTCCTGCTCCTGAATCCGTCACAAATGCTATTTGG
GAGATTTCCAAAAAGCTTACCAGCTATAAGATTATCAAAGACTTCCCAGA
ACTAGACTTGGGTACGATAGGCAAGAACAAGAAATACGGTCCATTACTCG
TTGACATTATCGATATTACAAAAGATTATGTCAACTTCTTGAAGGAAATC
TTCGATTTCGACTTAATCAAGAAATTCATCGATAATCAACGTTCTACTAA
GAATTGGAAGTTACTGTTTGACAGTATGAACGGTGTAACTGGACCATACG
GTAAGGCTATTTTCGTTGATGAATTTGGTTTACCGGCGGATGAGGTTTTA
CAAAACTGGCATCCTTCTCCGGATTTTGGTGGTATGCATCCAGATCCAAA
CTTAACTTATGCCAGTTCGTTAGTGAAAAGAGTAGATCGTGAAAAGATTG
AGTTTGGTGCTGCATCCGATGGTGATGGTGATAGAAATATGATTTACGGT
TACGGCCCATCTTTCGTTTCTCCAGGTGACTCCGTCGCAATTATTGCCGA
ATATGCAGCTGAAATCCCATATTTCGCCAAGCAAGGTATATATGGTCTGG
CCCGTTCATTCCCTACCTCAGGAGCCATAGACCGTGTTGCCAAGGCCCAT
GGTCTAAACTGTTATGAGGTCCCAACTGGCTGGAAATTTTTCTGTGCTTT
GTTCGACGCTAAAAAATTATCTATCTGTGGTGAAGAATCGTTTGGTACTG
GTTCCAACCACGTAAGGGAAAAGGACGGTGTTTGGGCCATTATGGCGTGG
TTGAACATCTTGGCCATTTACAACAAGCATCATCCGGAGAACGAAGCTTC
TATTAAGACGATACAGAATGAATTCTGAGCAAAGTACGGCCGTACTTTCT
TCACTCGTTATGATTTTGAAAAAGTTGAAACAGAAAAAGCTAACAAGATT
GTCGATCAATTGAGAGCATATGTTACCAAATCGGGTGTTGTTAATTCCGC
CTTCCCAGCCGATGAGTCTCTTAAGGTCACCGATTGTGGTGATTTTTCAT
ACACAGATTTGGACGGTTCTGTTTCTGACCATCAAGGTTTATATGTCAAG
CTTTCCAATGGTGCAAGATTCGTTCTAAGATTGTCAGGTACAGGTTCTTC
AGGTGCTACCATTAGATTGTACATTGAAAAATACTGCGATGATAAATCAC
AATACCAAAAGACAGCTGAAGAATACTTGAAGCCAATTATTAACTCGGTC
ATCAAGTTCTTGAACTTTAAACAAGTTTTAGGAACTGAAGAACCAACGGT
TCGTACTTAA.

### 3. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Experiments were conducted to investigate the performance of ligand-responsive gene expression systems that were previously investigated in S. cerevisiae in S. boulardii, and demonstrate their utility for several applications in engineered probiotics. SbGalt, a galactose-competent strain of S. boulardii, was constructed and activation of the pGAL1 galactose-inducible promoter was demonstrated. Dose-response behavior for this promoter was analyzed, in addition to four other inducible promoters (xylose, lactose or IPTG (Isopropyl ß-D-1-thiogalactopyranoside), copper, and anhydrotetracycline), establishing the first set of ligand-responsive expression systems in probiotic yeast. Results indicated that all 5 inducible systems demonstrated different gene activation ranges, and with non-toxic inducers that are largely absent from the human diet. The behavior of these promoters was investigated under anaerobic conditions similar to those found in the gut. Finally, results demonstrated the utility of these systems for yeast surface display and in vivo programmable gene expression with S. boulardii.

Repair of S. boulardii PGM2 gene enables simultaneous growth and induction on galactose. Experiments were conducted to first investigate the galactose-inducible promoter pGAL1 for the toolkit of inducible systems in S. boulardii. pGAL1 is one of the most commonly used promoters for inducible gene expression in S. cerevisiae due to its extensive characterization and high dynamic range. Also, as one of the monosaccharides comprising the lactose disaccharide, it is generally non-toxic to humans, except in the case of galactosemia, a rare genetic disorder that prevents galactose metabolism. Liu et al demonstrated that the PGM2 gene in S. boulardii MYA-796 harbors a point mutation that introduces a premature stop codon, leading to a truncated phosphoglucomutase enzyme and a very slow growth rate of S. boulardii on galactose. Liu et al demonstrated that when S. boulardii's PGM2 gene is reverted to the sequence found in S. cerevisiae, growth on galactose is restored; however, this study did not investigate the functionality of the pGAL1 promoter in either the wild-type strain or the strain with the PGM2 gene repaired (henceforth SbGal⁺).

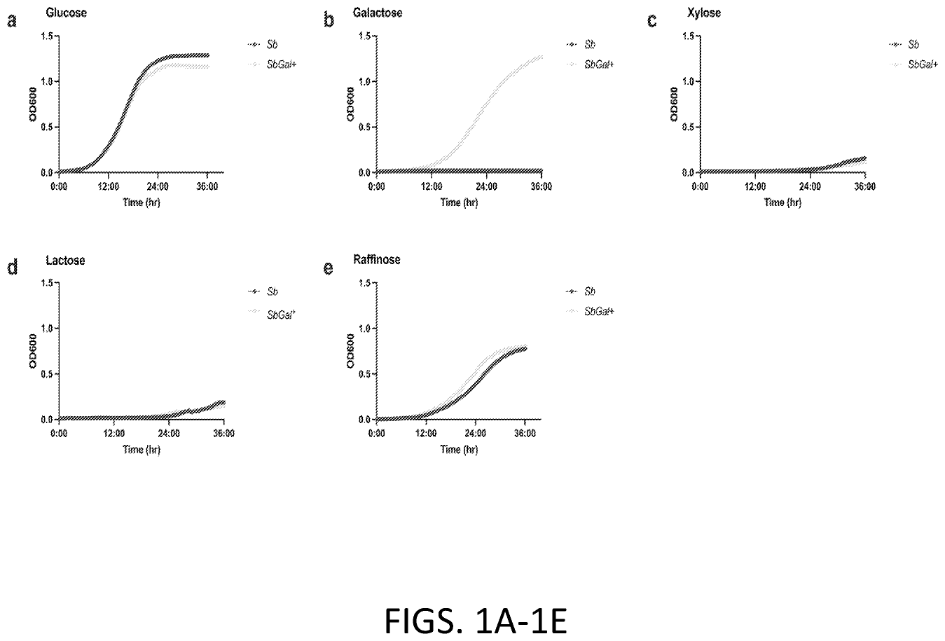

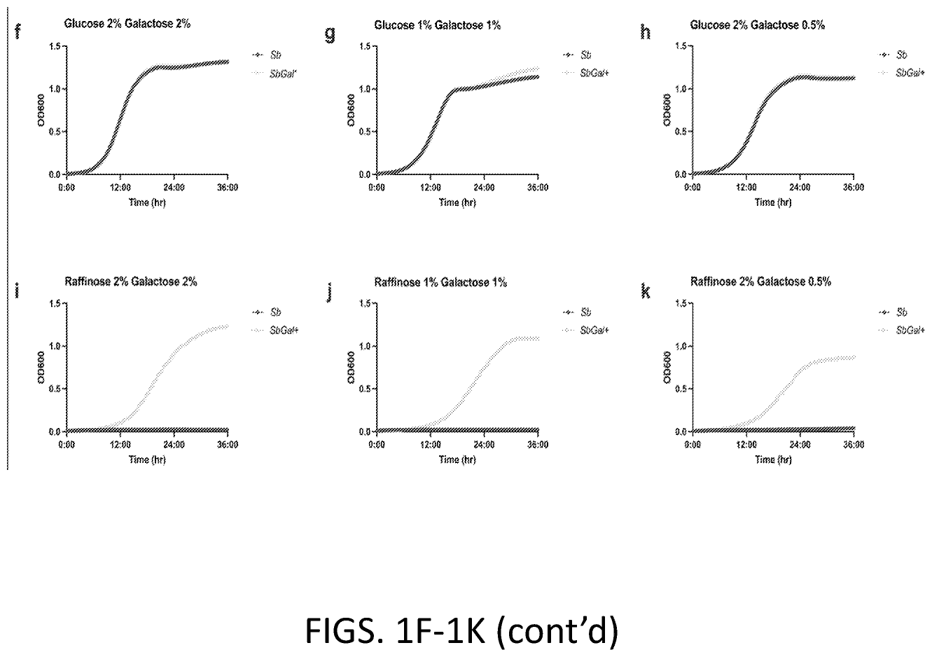

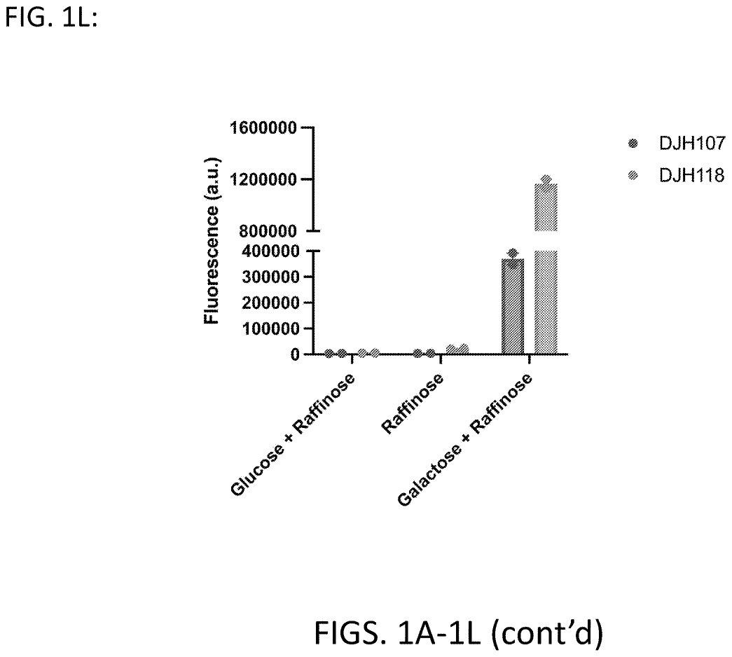

Growth and pGAL1 induction were investigated in both wild-type and SbGal⁺ strains. As shown in FIG. 1, wild-type S. boulardii grows very slowly on media with 2% galactose as the sole carbon source, while the SbGal⁺ strain grows at a much higher rate (FIGS. 1A and 1B), in agreement with Liu et al. To examine pGAL1 induction in these strains, a plasmid was constructed with production of yeast-enhanced green fluorescent protein (yeGFP) under the control of the pGAL1 promoter and transformed this plasmid to both wild-type and SbGal⁺ strains. Fluorescence of yeGFP was measured using flow cytometry. Both wild-type and SbGal⁺ strains exhibited induction of yeGFP fluorescence in the presence of galactose, demonstrating that although wild-type S. boulardii does not seem to efficiently metabolize galactose (FIG. 1B), it can still allow galactose-inducible gene expression (FIG. 1L) However, the inefficiency of galactose metabolism presents a problem with pGAL1-based gene expression in the wild-type strain, as another metabolizable carbon source must be provided. Glucose inhibits pGAL1 induction, so glucose cannot be used as a carbon source for wild-type S. boulardii when gene expression is desired. Therefore, the SbGal⁺ strain is desirable for pGAL1-based inducible gene expression in S. boulardii, as the repaired PGM2 gene product enables simultaneous growth and induction of pGAL1 on galactose.

### Example 2

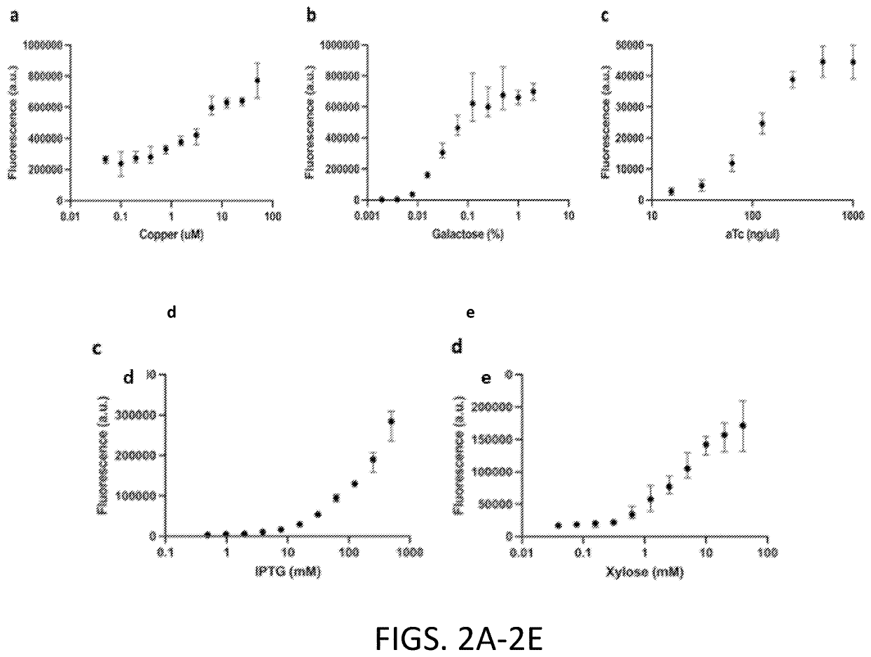

Characterization of dose-response relationships of inducible systems in S. boulardii. Having demonstrated inducible gene expression in S. boulardii using the pGAL1 promoter, experiments were conducted to investigate the dose-response relationship for this promoter, as well as other inducible systems used in S. cerevisiae. To expand the toolkit of inducible systems, the copper-inducible pCUP1 promoter (from MoClo Yeast Toolkit (YTK)) was used, as well as three engineered minimal promoters previously described: pTET (inducible by anhydrotetracycline (aTc)), PLAC (inducible by IPTG), and pXYL (inducible by xylose). All three of these promoters consist of two repressor-binding operator sequences, separated by spacers, placed upstream of a minimal ADH2 transcriptional start site. The yeGFP was placed under the control of each inducible promoter and transformed the resulting yeast expression vectors to the SbGal⁺ strain of S. boulardii. For those strains harboring the pTET, PLAC, and pXYL promoters, plasmids encoding the corresponding repressor proteins (tetR, lacI, and xylR, respectively) under the control of constitutive promoters were also introduced. After an overnight preculture in glucose media, each of the five strains were subinoculated into synthetic media with various concentrations of their respective inducers and grew for 20 hours before measuring fluorescence using the flow cytometer. Glucose was used as the carbon source for all strains except the pGAL1-yeGFP strain, in which raffinose was used as a carbon source. The pCUP1-yeGFP strain was grown in copper sulfate-free media to reduce nonspecific induction. All five systems showed increasing fluorescence with increasing inducer concentration, demonstrating ligand-responsive gene expression in S. boulardii (FIG. 2). The pCUP1 promoter exhibited the highest fluorescence level at low inducer concentration, matching previous reports that it is a “leaky” promoter.

### Example 3

Inducible systems enable tunable gene expression in an anaerobic environment. Engineering programmable gene expression in S. boulardii in vivo would enable it to deliver nutrients or therapeutics to the gut environment in a tunable fashion, expanding its utility as a “living medicine.” Due to their high fold-induction and high expression levels, experiments focused on the performance of pXYL, PLAC, and pGAL under gut-like conditions. Because the large intestine (where S. boulardii primarily resides) is characterized by very low oxygen concentrations, the performance of the selected inducible systems under was analyzed anaerobic conditions. yeGFP requires oxygen to fluoresce, so the fluorescent protein CaFBFP (Candida albicans-adapted flavin-based fluorescent protein) was selected for use as an anaerobic reporter of gene expression. Flavin-based fluorescent proteins do not require oxygen to fold. Production of CaFBFP was placed under the control of each inducible promoter in the library, transformed these plasmids to SbGal⁺ along with corresponding repressor plasmids as necessary, and cultivated the resulting strains under aerobic and anaerobic conditions. Cultures were cultivated aerobically overnight in glucose media and subinoculated 1:100 in media containing various concentrations of their respective inducers. The media pGAL1-CaFBFP strain also contained 2% raffinose as a carbon source. After a 24 hour induction period under either aerobic or anaerobic conditions, CaFBFP fluorescence was measured on a flow cytometer.

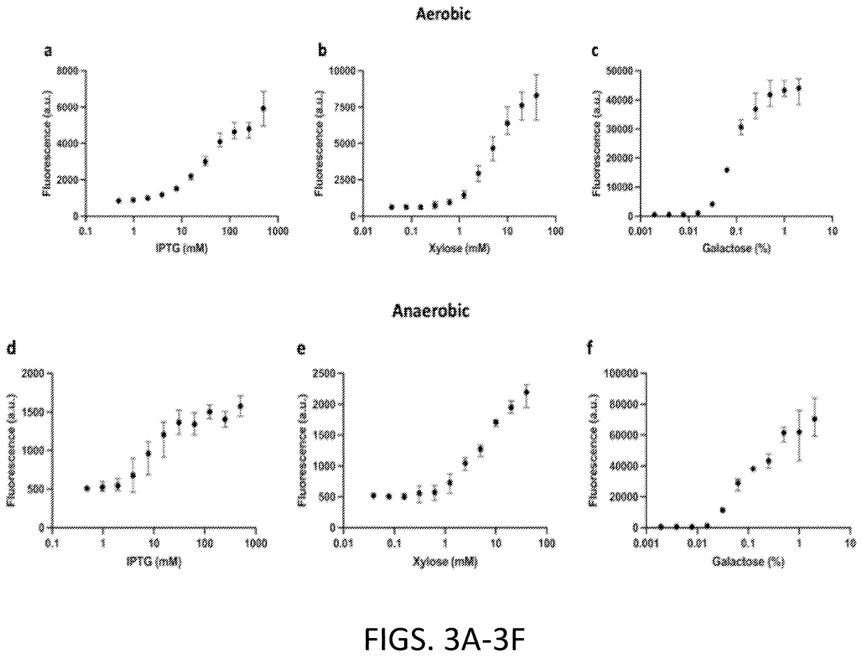

All three promoters demonstrated activation under anaerobic conditions (FIG. 3). Interestingly, pLAC and pXYL exhibited lower maximum activation under anaerobic conditions, while pGAL1 demonstrated higher maximum activation. This property, as well as the presence of galactose as a major component of human mucins, makes pGAL1 an attractive candidate for applications in the gut. Additionally, the CaFBFP dose-response curves for pLAC (under both aerobic and anaerobic conditions) showed a fluorescence signal approaching saturation with high concentrations of IPTG; this was not observed in the dose-response curve produced using yeGFP (FIG. 2). The concentration and fluorescence signal of flavin-based fluorescent proteins have previously been shown to decrease over time in E. coli cells growing exponentially, likely due to a depletion of flavin mononucleotide (FMN) necessary for fluorescence, as well as increased degradation of the proteins by proteases. It was hypothesized that the “early” saturation observed in CaFBFP dose-response curves is a consequence of this mechanism.

### Example 4

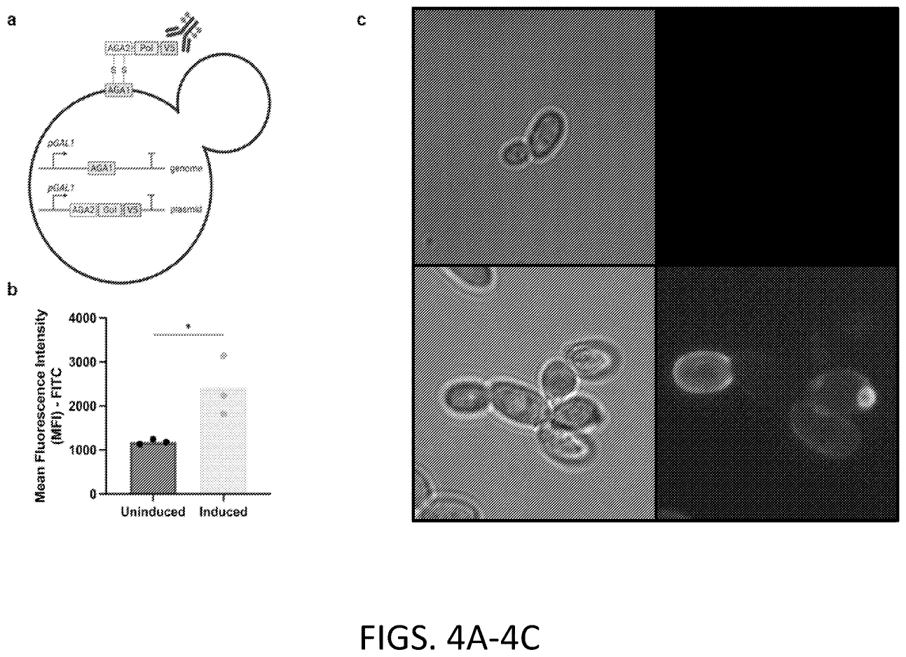

Inducible systems enable ligand-responsive surface display in S. boulardii. Having demonstrated inducible gene expression in S. boulardii under anaerobic conditions, experiments were conducted to investigate several possible applications for these inducible systems. As a case study, surface display was selected. Surface display of proteins on the cell surface of commensal bacteria has enabled enhanced colonization of the gut and localization of the bacteria to specific therapeutic sites. Therefore, enabling surface display in S. boulardii will add to its programmability as a therapeutic. In S. cerevisiae, surface display is enabled by fusing the protein of interest to Aga2. Upon expression of the fusion protein, it localizes to the cell wall surface through disulfide bonds between Aga1 and Aga2, resulting in display of the protein of interest. Following the design S. cerevisiae EBY100, a commonly used yeast strain for surface display, the cell surface anchor protein Aga1, was expressed in the genome using inducible pGAL1, and an Aga2-GFP fusion protein was expressed under inducible control of pGAL1 on a low-copy plasmid. GFP was selected as the protein of interest for surface display. Because expression of surface display proteins is toxic (FIG. 4B), inducible expression is preferable to constitutive expression as it enables the cell population to grow to the requisite size before initiating surface display.

### Example 5

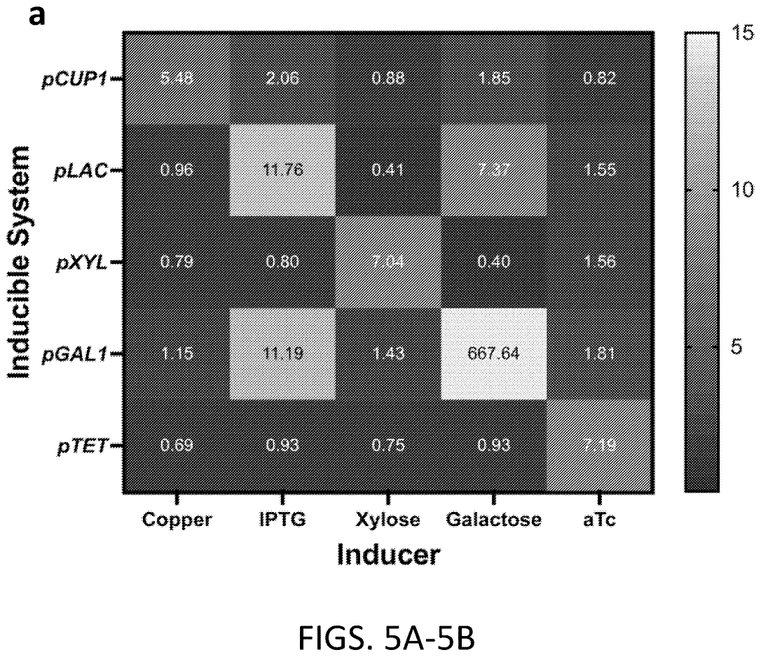

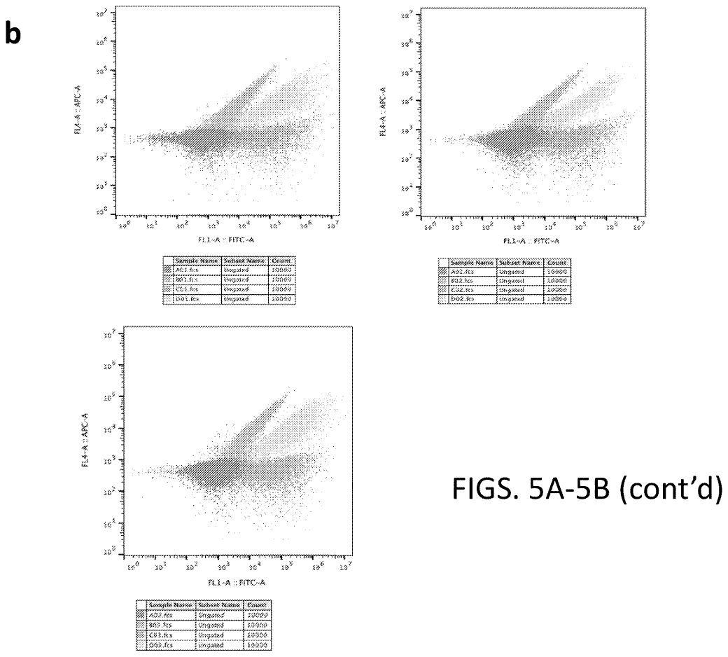

Inducible systems enable orthogonal gene expression. Engineered microbes can sometimes be programmed to make use of multiple inducible promoters each responding to different inducer molecules. Experiments were conducted to investigate whether any inducible promoters in the library responded to inducer molecules from other systems, as such “crosstalk” could present a barrier to engineering more complex circuit behavior. To check for crosstalk, yeast cultures harboring each inducible system were grown in cultures containing high concentrations of each of the five inducer molecules, as well as a no-inducer control. Flow cytometry was used to check for fluorescence in each culture. It was found that two inducible systems, the pGAL1 and PLAC system, exhibited significant crosstalk, with galactose inducing a 7.4-fold increase in pLAC expression and IPTG producing a 2-fold increase in pGAL1 expression (FIG. 5A). This lack of orthogonality most likely arises from the chemical similarity between the two inducer molecules. The pCUP1 promoter appears to exhibit 2-fold activation from IPTG and galactose as well, and aTc induces a small increase (˜1.5-fold) in the pLAC and pXYL systems. The pGAL1 system exhibited the highest fold-change (23-fold) in expression following induction with the intended ligand, while pCUP1 had the lowest (5.5-fold). This data suggests that although the five inducible systems are not fully orthogonal, several sets of two systems exist with minimal crosstalk between them, such as pCUP1 and pXYL, PLAC and pXYL, pTET and pCUP1, pXYL and pGAL1, and pGAL and pTET, with this final pair exhibiting the least amount of off-target repression or induction.

To further ensure that multiple inducible systems can operate simultaneously within the same cell, experiments were conducted to simultaneously express two different fluorescent proteins (yeGFP and mKate) using different inducible systems. yeGFP and mKate fluoresce at different wavelengths, enabling them to be detected separately by flow cytometry. For those systems requiring transcriptional repressors, single plasmids containing both a fluorescent protein and the cognate repressor of its promoter were constructed. This allowed co-transformation of two plasmids containing separate inducible systems, enabling simultaneous induction of any two inducible systems within the same cell. Each strain was exposed to four conditions: Inducer A, Inducer B, both inducers, and no inducers. Several pairs of inducible systems were capable of simultaneous orthogonal induction (FIG. 5B), demonstrating that this library of systems can be used to construct more complex circuits.

### Example 6

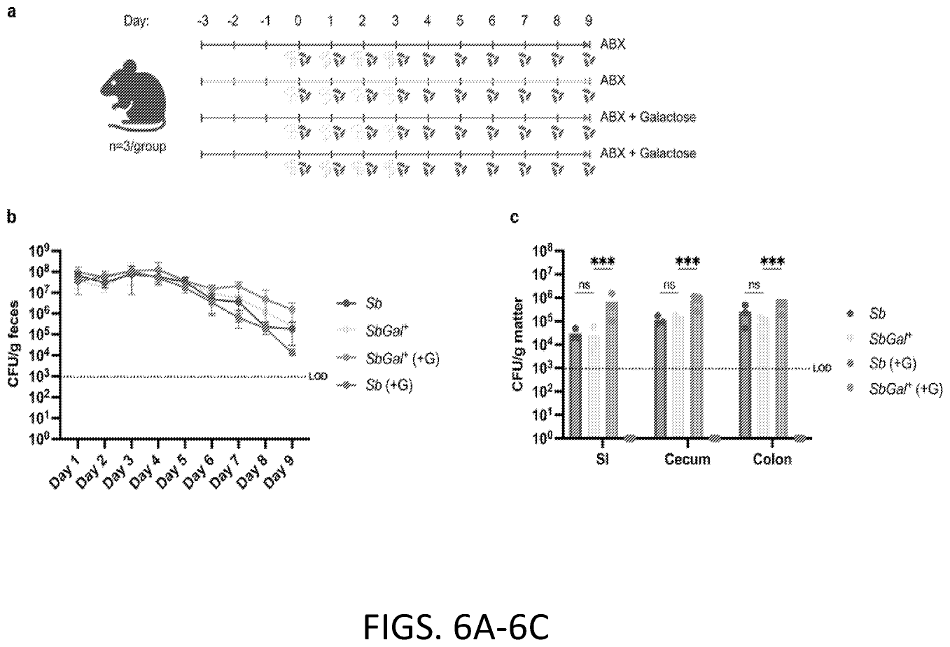

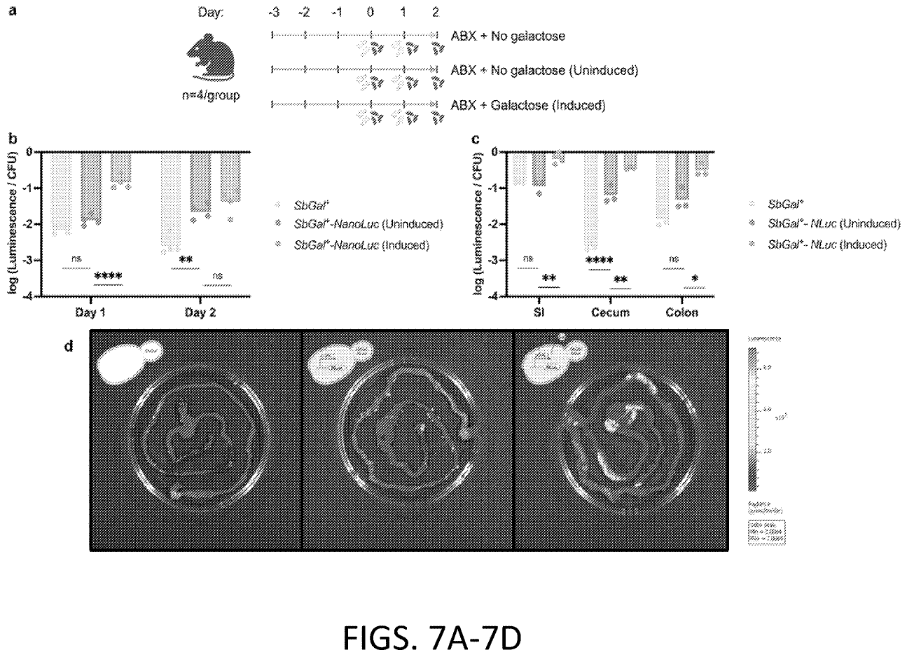

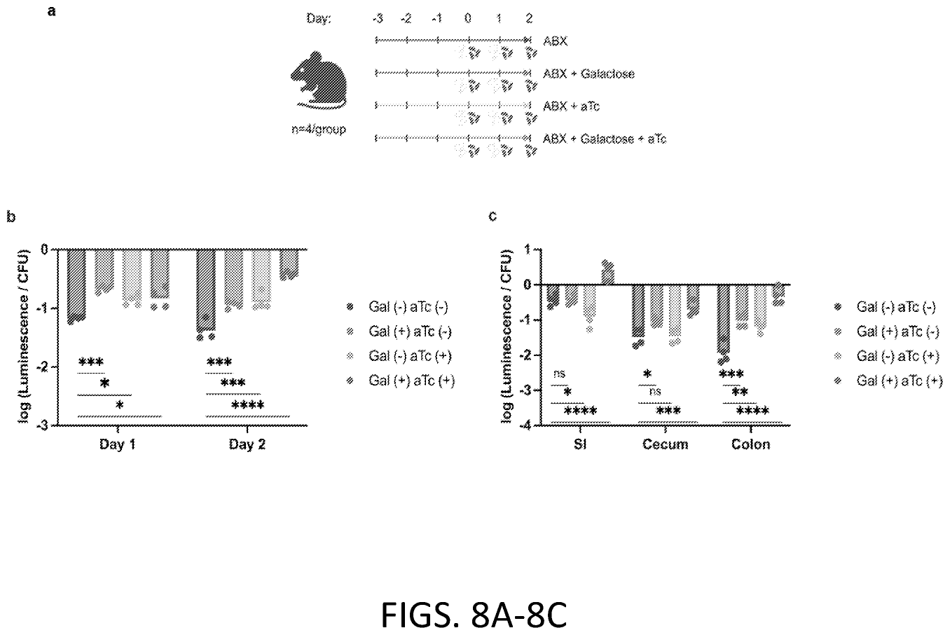

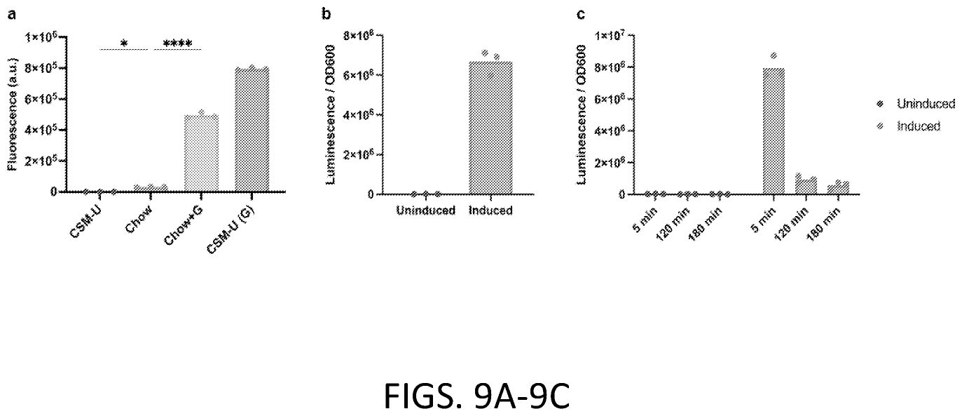

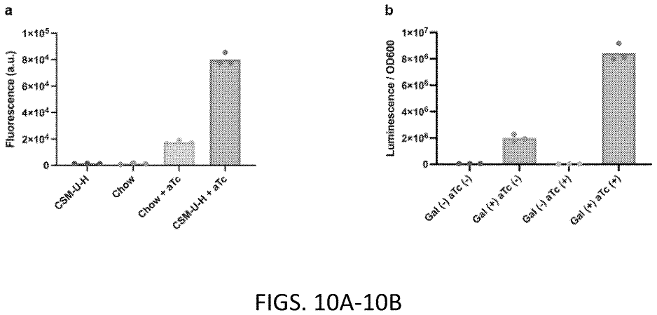

Galactose metabolism increases and prolongs gut colonization by Sb. Gut mucus is decorated with glycans that serve as a primary carbon source for many intestinal microbes. These glycans are primarily composed of galactose, N-acetylglucosamine, N-acetylgalactosamine, fucose, and sialic acid. Due to galactose's presence on intestinal mucus, it was hypothesized that the ability to metabolize galactose would provide a colonization advantage to SbGal⁺ versus wild-type Sb. To test this hypothesis, SbGal⁺ (DJH154) and wild-type Sb (ISA1151) were each delivered to 2 groups of antibiotic-treated mice. Both groups of mice consumed the noncaloric sweetener sucralose in their water(S), but one of these groups of mice also consumed galactose (S+G). After three consecutive days of Sb administration, fecal Sb levels were measured for one week. Subsequently, intestinal Sb levels were measured at the end of the experiment (day 9). It was observed that SbGal⁺ attained roughly one order of magnitude greater abundance in fecal material than wild-type Sb, and that co-administration of galactose further extended SbGal⁺'s residence time (FIG. 6). This increased colonization was reflected in the intestinal contents, with SbGal⁺ exhibiting higher levels of colonization in the small intestine, cecum, and colon, as compared to wild-type Sb. Remarkably, SbGal⁺ was recovered from the small intestine at levels comparable to the cecum and colon, in contrast to wild-type Sb, which was absent from the small intestine. The importance of the small intestine as the primary site of nutrient and drug absorption may enable future applications of engineered Sb therapeutics.

### Example 7

Inducible systems enable responsive in vivo protein production in mouse models. Recombinant proteins can be synthesized in situ in the mammalian gut using engineered probiotics. This cellular mechanism enables delivery of therapeutic and/or diagnostic proteins to the disease location providing sustainable treatment or tracking of the disease. In order to test whether GALI promoter can be used and induced to produce recombinant proteins in the mouse gut, constructed a SbGal+ strain with a plasmid encoding for nanoluciferase (NLuc) protein under the control of GALI promoter (SbGal+-NLuc). SbGal+-NLuc strain was functional and able to produce nanoluciferase when cultured in media (CSM-U) supplemented with galactose. The detection of nanoluciferase produced by SbGal+-NLuc in supernatant samples was at its optimum 5 minutes after assay development. In addition, the SbGal+ strain expressing yeGFP was cultured under the control of pGAL1 in media prepared from chow diet pellets with or without addition of galactose to test whether the diet would inhibit and/or achieve pGAL1. pGAL1 activation was observed when the cells were cultured in chow diet media only, however this activation was 15.5-fold higher when galactose was supplemented with the media, indicating that pGAL1 induction will be possible in the mouse gut when galactose is provided. 3 groups of antibiotic-treated mice were given different SbGal+ strains or exposed to different sugar induction in water for 2 days (Day 0-Day 1).

The first set of mice were given SbGal+ strain with the noncaloric sweetener sucralose in their water (control group, background luminescence). The second set of mice were given SbGal+-NLuc with the noncaloric sweetener sucralose in their water (no induction control group). The third set of set of mice were given SbGal+-NLuc with the noncaloric sweetener sucralose and galactose as inducer in their water. Daily feces were collected for CFU enumeration and nanoluciferase activity detection via luminescent readout. On the last day of the experiment (day 2), intestinal tissues were collected for CFU enumeration, nanoluciferase activity detection and imaging. All luminescence values were normalized by CFU. Feces obtained from mice given SbGal+-NLuc and consumed galactose had higher luminescence values compared to the control groups, indicating higher NLuc presence in the feces. Fecal samples from mice given SbGal+-NLuc but no galactose consumption (induction control), had higher luminescence values from the control group mice, indicating galactose present in the mucus and mice diet is also enough for activation of the NLuc production but this can be enhanced with additional supplementation of the inducer in the water. This trend was also observed in the tissue samples. Mice given SbGal+-NLuc and consumed galactose had more NLuc in all three regions of lower gastrointestinal tract (small intestine, cecum, and colon) compared to the two control groups. Similarly, mice given SbGal+-NLuc but no galactose consumption (induction control) were able to synthesize NLuc in the gastrointestinal tract, mostly in the cecum. Bioluminescence imaging of the tissue samples confirmed that the hotspots for NLuc production upon galactose induction were in small intestine and cecum followed by colon.

### Example 8

Inducible systems enable in vivo logical operations in the mouse gut. Using logic gates in engineered probiotics allows for precise control over protein synthesis in the gut. This can lead to more effective and targeted treatments for a variety of conditions, with fewer side effects compared to systemic drug administration. In order to test this hypothesis, a transcriptional AND gate was constructed in which both galactose and aTc are necessary for activation of transcription, by cloning two Tet operators (separated by a T-rich spacer) upstream of the pGAL1 promoter, which contains an operator for the Gal4p activator protein. In the absence of aTc, the TetR repressor is bound to the Tet operator sites, preventing readthrough, while in the absence of galactose, the Gal4p activator is absent, preventing transcription. Only when both inducers are present should transcription of downstream genes occur. Nanoluciferase (NanoLuc) was selected as the reporter for AND gate behavior. Gene for Tet repression was placed under control of its corresponding constitutive promoter and cloned into a plasmid with HIS3 marker.

The AND gate-NanoLuc unit was cloned into a plasmid with URA3 marker and these plasmids were transformed into the SbGal⁺ (SbGal⁺-pGALTET-NLuc). SbGal⁺-pGALTET-NLuc was functional and able to produce nanoluciferase when cultured in media (CSM-U-H) supplemented with galactose and aTc. Some limited activation of the AND-gate was observed when only galactose was present, however not to the extent of when both inducers were present. In addition, activation of Tet operation by chow diet was tested in a similar way it was tested in galactose operation. SbGal+ strain expressing yeGFP under the control of pTET promoter was cultured in media made from chow diet pellets with or without addition of aTc. Only when aTc was present in the chow diet media, yeGFP was produced. 4 groups of antibiotic-treated mice were given different SbGal⁺-pGALTET-NLuc and exposed to induction conditions in water for 2 days (Day 0-Day 1). First set of mice were given SbGal⁺-pGALTET-NLuc strain with the noncaloric sweetener sucralose in their water (Gal-aTc-). Second set of mice were given SbGal⁺-pGALTET-NLuc with the noncaloric sweetener sucralose and inducer galactose in their water (Gal+aTc-). Third set of set of mice were given SbGal⁺-pGALTET-NLuc with the noncaloric sweetener sucralose and inducer aTc in their water (Gal-aTc+). Fourth set of set of mice were given SbGal⁺-pGALTET-NLuc with the noncaloric sweetener sucralose and inducers galactose and aTc in their water (Gal+aTc+). Daily feces were collected for CFU enumeration and nanoluciferase activity detection via luminescent readout. On the last day of the experiment (day 2), intestinal tissues were collected for CFU enumeration and nanoluciferase activity detection and imaging. All luminescence values were normalized by CFU. Feces and tissues obtained from mice given SbGal⁺-pGALTET-NLuc and administered galactose and aTc exhibited the most nanoluciferase activity compared to single inducer or no inducer groups, the highest AND-gate activation being in the small intestine. Single inducer groups exhibited some level of AND-gate activation due to availability of galactose in the diet and/or in the mucus or strength of galactose operation sidetracking Tet repression (as observed in the in vitro testing), however the maximum activation was possible when both inducers present in vivo.

1. An engineered yeast cell comprising:
(i) at least one genetic modification that produces a functional phosphoglucomutase (PGM2) protein; and
(ii) at least one exogenous polynucleotide operably linked to a pGAL1 promoter;
wherein expression of a target polypeptide or protein encoded by the exogenous polynucleotide is increased compared to a yeast cell lacking the at least one genetic modification.

2. The engineered yeast cell of claim 1, wherein the at least one genetic modification comprises a mutation in the gene encoding PGM2.

3. The engineered yeast cell of claim 1, wherein the at least one genetic modification comprises a point mutation at position 1276, 1277, and/or 1278 in the gene encoding PGM2.

4. The engineered yeast cell of claim 3, wherein the point mutation is:
(i) T1276A, T1276C, or T1276G;
(ii) G1277C or G1277T; or
(iii) A1278T, A1278C, or A1278G.

5. The engineered yeast cell of claim 1, wherein the yeast cell is selected from the group consisting of S. boulardii, S. cerevisiae, P. pastoris, Y. lipolytica, C. albicans, K. marxiamus, Debaryomyces hansenii, and Kluyveromyces lactis.

6. The engineered yeast cell of claim 1, wherein the at least one genetic modification enables the yeast cell to utilize galactose as a carbon source.

7. The engineered yeast cell of claim 1, wherein the at least one genetic modification enhances colonization in a host.

8. The engineered yeast cell of claim 1, wherein the at least one genetic modification enhances colonization in a host in the presence of galactose.

9. The engineered yeast cell of claim 1, wherein the at least one genetic modification activates the pGAL1 promoter.

10. The engineered yeast cell of claim 1, wherein expression of the target polypeptide or protein encoded by the exogenous polynucleotide is increased compared to a yeast cell lacking the at least one genetic modification.

11. The engineered yeast cell of claim 1, further comprising at least one gene knockout, wherein expression and/or secretion of the target polypeptide or protein is increased at least 1.2-fold compared to a yeast cell lacking the at least one gene knockout.

12. The engineered yeast cell of claim 11, wherein the at least one genetic knockout comprises knockout of YPS1, PRB1, PEP4, and/or APE1.

13-16. (canceled)

17. The engineered yeast cell of claim 1, wherein the exogenous polynucleotide comprises a secretion signal upstream of the target polypeptide or protein.

18. The engineered yeast cell of claim 17, wherein the secretion signal comprises an alpha mating factor secretion signal, an invertase secretion signal, a YAP3-TA57 secretion signal, a preOST1-proαMF (I) secretion signal, or a preOST1-proαMF (MUT1) secretion signal, and any combinations thereof.

19. The engineered yeast cell of claim 1, wherein the yeast cell is from an S. boulardiiΔura3 strain (DD277), an S. boulardii Δura3 Δhis3 (DD313) strain, an S. boulardiiΔura3 Δhis3PGM2 (DJH077) strain, a MYA-796 strain, or a MYA-797 strain.

20. A composition comprising the engineered yeast cell of claim 1.

21-27. (canceled)

28. A method of growing a yeast cell in media comprising galactose, the method comprising:
making at least one genetic modification to the yeast cell such that the yeast cells is capable of producing a functional phosphoglucomutase (PGM2) protein;
wherein the yeast cell is a Saccharomyces boulardii cell; and
wherein the at least one genetic modification enables the yeast cell to utilize galactose as a carbon source.

29. The method of claim 28, wherein the method further comprises inducing expression of an exogenous polypeptide or protein via activation of a pGAL1 promoter.

30. (canceled)

31. A method of enhancing colonization of a yeast cell in a host, the method comprising:
administering a composition comprising an engineered yeast cell that comprises at least one genetic modification that produces a functional phosphoglucomutase (PGM2) protein;
wherein the yeast cell is a Saccharomyces boulardii cell; and
wherein the at least one genetic modification enables the yeast cell to utilize galactose as a carbon source.

32. The method of claim 31, wherein the engineered yeast cell further comprises at least one exogenous polynucleotide operably linked to a pGAL1 promoter.

33-35. (canceled)


SEQUENCE LISTING (1 sequence)

SEQ ID NO: 1 - DNA, 1710 bp, Saccharomyces boulardii
  source 1..1710: mol_type = other DNA
atgtcatttcaaattgaaacggttcccaccaaaccatatgaagaccaaaagcctggtacctctggtttgcgtaagaagacaaaggtgtttaaagacgaacctaactacacagaaaatttcattcaatcgatcatggaagctattccagagggttctaaaggtgccactcttgttgtcggtggtgatgggcgttactacaatgatgtcattcttcataagattgccgctatcggtgctgccaacggtattaaaaagttagttattggtcagcatggtcttctgtctacgccagccgcttctcacatcatgagaacctacgaggaaaaatgtactggtggtattatcttaaccgcctcacataatccaggtggtccagaaaatgacatgggtattaagtataacttatccaatgggggtcctgctcctgaatccgtcacaaatgctatttgggagatttccaaaaagcttaccagctataagattatcaaagacttcccagaactagacttgggtacgataggcaagaacaagaaatacggtccattactcgttgacattatcgatattacaaaagattatgtcaacttcttgaaggaaatcttcgatttcgacttaatcaagaaattcatcgataatcaacgttctactaagaattggaagttactgtttgacagtatgaacggtgtaactggaccatacggtaaggctattttcgttgatgaatttggtttaccggcggatgaggttttacaaaactggcatccttctccggattttggtggtatgcatccagatccaaacttaacttatgccagttcgttagtgaaaagagtagatcgtgaaaagattgagtttggtgctgcatccgatggtgatggtgatagaaatatgatttacggttacggcccatctttcgtttctccaggtgactccgtcgcaattattgccgaatatgcagctgaaatcccatatttcgccaagcaaggtatatatggtctggcccgttcattccctacctcaggagccatagaccgtgttgccaaggcccatggtctaaactgttatgaggtcccaactggctggaaatttttctgtgctttgttcgacgctaaaaaattatctatctgtggtgaagaatcgtttggtactggttccaaccacgtaagggaaaaggacggtgtttgggccattatggcgtggttgaacatcttggccatttacaacaagcatcatccggagaacgaagcttctattaagacgatacagaatgaattctgagcaaagtacggccgtactttcttcactcgttatgattttgaaaaagttgaaacagaaaaagctaacaagattgtcgatcaattgagagcatatgttaccaaatcgggtgttgttaattccgccttcccagccgatgagtctcttaaggtcaccgattgtggtgatttttcatacacagatttggacggttctgtttctgaccatcaaggtttatatgtcaagctttccaatggtgcaagattcgttctaagattgtcaggtacaggttcttcaggtgctaccattagattgtacattgaaaaatactgcgatgataaatcacaataccaaaagacagctgaagaatacttgaagccaattattaactcggtcatcaagttcttgaactttaaacaagttttaggaactgaagaaccaacggttcgtacttaa